(19) United States
(12) Patent Application Publication
(10) Pub. No.: US 2008/0166433 A1
(43) Pub. Date: Jul. 10, 2008

(54) Methods and Composition For Dietary Supplements

(75) Inventors: Dusan Miljkovic, San Diego, CA (US); Jovan Hranisavljevic, Belgrade (YU)

Correspondence Address: FISH & ASSOCIATES, PC;ROBERT D. FISH, 2603 Main Street, Suite 1050, Irvine, CA, 92614-6232, US

(73) Assignee: VDF FUTURECEUTICALS, INC., Momence, IL (US)

(21) Appl. No.: 12/043,102

(22) Filed: Mar. 5, 2008

Related U.S. Application Data
(62) Division of application No. 10/220,761, filed on Dec. 16, 2002.
(60) Provisional application No. 60/187,641, filed on Mar. 8, 2000.

(51) Int. Cl. A61K 36/00

(52) U.S. Cl. 424/725

(57) ABSTRACT

Compositions and methods are provided to reduce glucose concentration in an organism. Particularly contemplated compositions include a compound that binds to a thaumatin-like protein and that is isolated from a plant. Contemplated compositions further reduce blood lipid concentrations at the concentration effective to reduce the glucose concentration.

### FIELD OF THE INVENTION

The field of the invention is dietary supplements and related methods.

### BACKGROUND OF THE INVENTION

Elevated blood glucose and blood lipids are a relatively common underlying condition in numerous diseases and may be acquired in various ways. Among other causes, elevated blood glucose levels is frequently precipitated by an altered metabolism associated with a diabetic condition, and treatment of diabetic conditions often includes insulin therapy along with synthetic oral anti-diabetic agents, such as metformin, sulfonylurea, etc. Despite an improvement of some clinical parameters (i.e. reduction of blood glucose to at least some extent) in people with elevated blood lipid and blood glucose, various side effects, including insulin resistance, allergic reactions, etc. may arise from long-term treatment using insulin.

Alternative treatments of diabetes, and especially non-insulin dependent diabetes mellitus (NIDDM), are frequently based on yeast, or derivatives of yeast. Yeast can be grown in the presence of chromium salts, and yeast cells or extracts of cells grown in that manner are particularly rich in “glucose tolerance factor” (GTF), a compound known to enhance the biological effect of insulin. Although some yeast preparations help reducing elevated blood glucose concentrations, in many cases considerable amounts of yeast preparations must be ingested for a substantial period in order to improve a hyperglycemic condition. Moreover, long-term use of yeast preparations over extended periods tends to become problematic for some patients, especially where those patients have a history of yeast infections. Still further, many crude yeast preparations have a bitter taste that some patients may find objectionable.

To alleviate at least some of the problems associated with yeast preparations, concentrated, de-bittered and freeze dried yeast preparations have been developed. Such preparations are typically in tablet form, and may conveniently be ingested during a meal. However, the relatively high degree of processing of such cells/extracts may reduce the biological potency of the yeast preparation. Moreover, preservatives and additives (e.g., for pressing or otherwise forming of tablets) are typically needed to maintain at least some anti-hyperglycemic activity.

In still other methods of reducing blood glucose on a non-insulin basis, chromium picolinate may be administered. Chromium picolinate is reported to be moderately effective in reducing an elevated blood glucose level in human. However, chromium picolinate exhibits considerable toxicity and may therefore not be generally regarded as safe.

Although various methods of reducing an increased blood concentration of glucose are known in the art, all or almost all of them suffer from one or more disadvantages. Therefore, there is still a need to provide improved compositions and methods to reduce glucose concentration.

### SUMMARY OF THE INVENTION

The present invention is directed to compositions and methods of reducing glucose concentrations in an organism. More specifically, contemplated compositions comprise a compound that binds to a thaumatin-like protein and reduces a concentration of glucose in an organism when the compound is administered to the organism at a concentration effective to reduce the concentration of glucose.

In one aspect of the inventive subject matter, the compound is isolated from a plant, preferably a plant belonging to the family of Poaceae, and most preferably from Hordeum vulgare. Contemplated isolation procedures include malting, mashing, salt extraction, buffer extraction, ethanol extraction, anion exchange chromatography, and molecular sieving. Alternatively, contemplated compounds may be synthesized de-novo at least in part.

In another aspect of the inventive subject matter, contemplated compounds are hydrophobic, have a molecular weight of no more than 1000 Da, are soluble in a lipophilic solvent at a concentration of at least 10 mg per milliliter, and have a UV/VIS absorption maximum of about 260 nm. In especially preferred aspects, the composition further reduces the concentration of a blood lipid (e.g., triglycerides, fatty acids, HDL-cholesterol, and LDL cholesterol), and in still further aspects of the inventive subject matter, the composition may further comprise a tocol, vitamins, or other dietary supplements which may or may not be active in regulation of blood glucose and/or blood lipids.

In a further aspect of the inventive subject matter, a method of reducing a glucose concentration in an organism comprises a step in which a composition is provided that includes a compound that binds to a thaumatin-like protein. In another step, contemplated compositions are administered to the organism in a dosage effective to decrease the concentration of glucose.

Various objects, features, aspects and advantages of the present invention will become more apparent from the following detailed description of preferred embodiments of the invention.

### BRIEF DESCRIPTION OF THE DRAWING

FIG. 1 is a flow diagram showing an exemplary method of reducing blood concentration of glucose according to the inventive subject matter.

FIG. 2 is a schematic showing an exemplary preparation of contemplated compounds and thaumatin-like proteins.

FIG. 3A is a table depicting reduction of blood glucose concentrations in human volunteers using contemplated compositions according to the inventive subject matter.

FIG. 3B is another table depicting reduction of blood glucose concentrations in human volunteers using contemplated compositions according to the inventive subject matter.

FIG. 4A is a table depicting reduction of blood lipid concentrations in human volunteers using contemplated compositions according to the inventive subject matter.

FIG. 4B is another table depicting reduction of blood lipid concentrations in human volunteers using contemplated compositions according to the inventive subject matter.

FIG. 5 is a graph depicting fermnentation rates of yeast incubated with contemplated compounds at anaerobic and aerobic conditions.

### DETAILED DESCRIPTION

As used herein the term “compound that binds to a thaumatin-like protein” refers to any compound or mixture of compounds that exhibit a binding preference to a thaumatin-like protein from barley of at least 10-fold, more preferably at least 100-fold over binding to other barley proteins, wherein binding of contemplated compounds to the thaumatin-like protein will preferably have a K_{D} of less than 10⁻³M, more preferably of less than 10⁻⁴M. The mode of binding need not be limited to a single interaction (e.g., hydrophobic interaction), but may include multiple interactions (e.g., electrostatic interactions and hydrogen bonding, etc.). It is especially contemplated that binding is reversible, however, irreversible binding is not excluded. Although thaumatin-like proteins from barley are generally preferred binding partners for compounds according to the inventive subject matter, thaumatin-like proteins from alternative sources, including microorganisms, plants, and animals are also contemplated. Thaumatin-like proteins are a well characterized class of polypeptides and are described, for example, in Cvetkovic et al., J. Serb Chem. Soc. 62(9):777-786 (1997), Cvetkovic et al., J. Serb. Chem. Soc. 62(1):51-56 (1997) and Cvetkovic et al., J. Inst. Brew. 103:183-186 (1997), all of which are incorporated by reference herein.

As also used herein, the term “elevated glucose concentration” refers to a concentration that is above the clinical range considered normal (i.e., above 110 mg/dl). Similarly, the term “elevated lipid concentration” refers to a concentration of blood lipids that is above the clinical range considered normal.

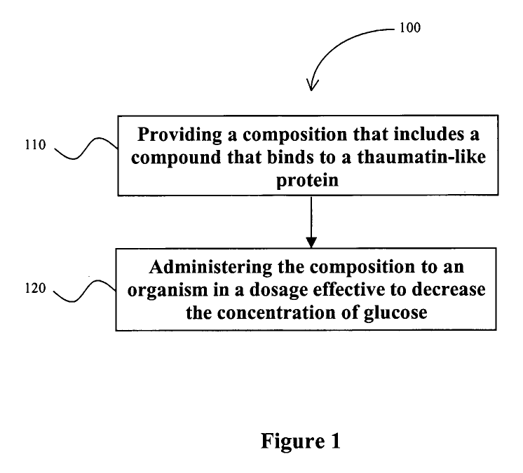

In FIG. 1, a method 100 of reducing a glucose concentration in an organism has a step 110 in which a composition is provided that includes a compound that binds to a thaumatin-like protein. In a subsequent step 120, the composition is administered to the mammal in a dosage effective to decrease the blood concentration of glucose.

In an especially preferred aspect of the inventive subject matter, the composition is prepared from Hordeum vulgare (as outlined in examples, infra), and orally administered in 3 daily doses of 500 mg, respectively, to a human diagnosed with non-insulin dependent diabetes mellitus (NIDDM). Thus, especially preferred compositions include a compound that binds to thaumatin-like proteins and that reduces a concentration of glucose in an organism when the compound is administered to the organism at a concentration effective to reduce the concentration of glucose.

In alternative aspects of the inventive subject matter, it is contemplated that appropriate compositions and compounds need not be limited to a preparation from Hordeum vulgare, but may also include preparations from various plants other than Hordeum vulgare, and particularly contemplated alternative plants include Hordeum spec., and members of the poaceae family. While the preparation of contemplated compositions and/or compounds is preferably from plant extracts, it should further be appreciated that contemplated compositions and/or compounds may also be isolated from microorganisms (i.e., bacteria, fungi, yeasts, unicellular eucaryotic organisms) or animals, so long as contemplated compounds bind to a thaumatin-like protein and reduce a glucose concentration in an organism.

In still further alternative aspects, it should be appreciated that contemplated compounds may be isolated, purified to homogeneity, and the structure be elucidated. Consequently, it should be appreciated that contemplated compounds and/or compositions may be entirely (de novo) or partially synthesized/modified in vitro. For example, where contemplated compounds are partially synthesized, a precursor of contemplated compounds may be isolated from a plant or microorganism, and then be subjected to one or more steps to arrive at contemplated compounds. Alternatively, contemplated compounds may be modified in one or more synthetic steps to impart a particularly desirable physico-chemical property. For example, contemplated compounds may be esterified with a polar compound (e.g, polyethylene glycol) to increase water solubility. In another example, contemplated compounds may be coupled to a resin or other material to control the rate of release to the organism.

Preferred contemplated compounds have a relatively low molecular weight, typically no more than 1000 Da, however, it should be recognized that the molecular weight may vary considerably and will predominantly depend on the source from which the compound is isolated, synthetic modifications, dimerizations and multimerizations. Likewise, it is contemplated that suitable compounds need not be limited to compounds having a UV absorption maximum at about 260 nm (which is characteristic for contemplated compounds isolated using the procedure outlined below), and various spectral characteristics other than a UV₂₆₀ peak are also suitable. Similarly, while contemplated compounds isolated from Hordeum vulgare are soluble in a lipophilic solvent at a concentration of at least 10 mg per milliliter, higher or lower solubilities are also contemplated and will typically depend on the source from which contemplated compounds are isolated, and/or on further chemical modifications of contemplated compounds. The term “lipophilic solvent” as used herein includes all solvents that have a miscibility with H₂O of less than 10 vol %.

While it is generally preferred that contemplated compounds are chemically substantially pure (i.e., concentration of contemplated compounds greater than 90 wt %, preferably greater than 95 wt %, most preferably greater than 99 wt %), it should also be appreciated that contemplated compounds may be coupled to one or more than one molecule, and particularly contemplated molecules include thaumatin-like proteins. Thus, contemplated compositions include complexes between contemplated compounds and thaumatin-like proteins, and especially include complexes between contemplated compounds and thaumatin-like proteins as they are isolated from the appropriate sources (infra).

With respect to the glucose concentration, it is generally contemplated that the glucose concentration is a blood glucose concentration. However, further contemplated glucose concentrations also include concentrations of glucose covalently or non-covalently bound to molecules found within the organism, and especially contemplated alternative glucose concentrations include concentrations of glycosylated proteins (e.g., glycosylated hemoglobin or collagen).

While it is generally contemplated that suitable thaumatin-like proteins are isolated from Hordeum vulgare, alternative thaumatin-like proteins are also contemplated and include thaumatin-like proteins isolated from microorganisms, plants, and animals, which may or may not be expressed in a recombinant system. There are various protocols for isolation of thaumatin-like proteins known in the art (see e.g., Barre et al, Purification and structural analysis of an abundant thaumatin-like protein from ripe banana fruit. Planta. 2000 November;211(6):791-9; Oh, et al., Isolation of a cDNA encoding a 31-kDa, pathogenesis-related 5/thaumatin-like (PR5/TL) protein abundantly expressed in apple fruit. Biosci Biotechnol Biochem. 2000 February;64(2):355-62; Tattersall, et al. Identification and characterization of a fruit-specific, thaumatin-like protein that accumulates at very high levels in conjunction with the onset of sugar accumulation and berry softening in grapes. Plant Physiol. 1997 July;114(3):759-69), and all the known protocols are considered suitable for use in conjunction with the teachings presented herein.

It should be especially appreciated that contemplated compositions not only reduce elevated blood glucose concentration in human suffering from NIDDM, but may also reduce blood glucose concentrations in individuals having elevated blood glucose concentrations for reasons other than NIDDM, including obesity, dietary effects, etc. It is especially contemplated that individuals with or without NIDDM will have a blood glucose concentration of at least 90 mg/dl, more preferably of at least 120 mg/dl, and most preferably of at least 200 mg/dl.

Furthermore, contemplated compositions have also been shown to advantageously reduce elevated blood lipid concentrations (infra), wherein blood lipids particularly include triglycerides, fatty acids, HDL-cholesterol, and LDL-cholesterol, and it is further contemplated that the reduction of blood lipids may be concomitantly with the reduction of blood glucose levels, or independent of the reduction of the blood glucose level.

In further aspects of the inventive subject matter, it should be appreciated that contemplated compositions may further comprise active or inactive ingredients, including compositions known to decrease a blood lipid concentration, and/or compositions known to decrease blood sugar concentrations. For example, alternative compositions may include at least one of a tocol, vitamins, and/or mineral preparations, GTF, metformin, sulfonylurea, and the like. Inactive ingredients include fillers, coloring agents, stabilizers, and the like.

Thus, an exemplary method of treating a person (e.g., diagnosed with NIDDM) having an increased blood concentration of glucose of approximately 150 mg/dl, and an increased blood concentration of total cholesterol of above 280 mg/dl, or more has one step in which contemplated compositions are provided. In a further step, the composition is administered to the person in a dosage effective to decrease the concentration of glucose.

With respect to the blood glucose level it is contemplated that a treatment according to the inventive subject matter need not be limited to blood glucose levels of approximately 150 mg/dl, but may also be indicated at many blood concentrations of glucose above 70-110 mg/dl. Although not wishing to be bound to a particular theory or mechanism, it is contemplated that the reduction in the blood glucose level may be due to an enhanced glucose uptake into the cell. However, it should be noted that compositions according to the inventive subject matter are non-GTF compositions. The duration for contemplated treatments may vary significantly, and suitable durations may be within the range of a single dose, but also for a predetermined period, including one week, several weeks, several months, and even several years. Consequently, it be appreciated that compositions according to the inventive subject matter may also be prophylactically administered to a human to prevent hyperglycemia, or some form of dyslipidemia.

In further alternative aspects of the inventive subject matter, the composition may also be administered to an organism other than a human, and particularly preferred alternative organisms include livestock (e.g., cattle, pigs, horses, etc.) and pets (e.g, dogs, cats, rodents, birds, etc.). With respect to contemplated compositions, the same considerations as described above apply.

It is especially contemplated that treatment according to the inventive subject matter may also result in significant weight loss, particularly in persons with obesity, NIDDM, or other condition associated with increased body weight. It is generally contemplated that the treatment according to the inventive subject matter is not limited to reduction of blood glucose alone, but may concomitantly (or by itself) include reduction of a particular lipid or lipid group. For example, slightly elevated total cholesterol (e.g., 220 mg/dl) may be an indication for treatment with the contemplated compounds. Alternatively, it is contemplated that an imbalance between HDL and LDL (i.e. LDL>>HDL) may be normalized employing a treatment according to the inventive subject matter. Similarly, while the total cholesterol in the patient need not be elevated, treatment with the contemplated method may still be indicated due to an elevated triglyceride level.

With respect to the dosage, form, and route of administration it is contemplated that there are many alternative oral preparations besides 3 oral daily doses of 500 mg. For example, where relatively high dosages are required, dosages may increase from 500 mg-5 g per day, and more. High dosages may also be required where the potency of an extract is relatively low. Likewise, in cases where low dosages (e.g., maintenance therapy) are required, or the extract has a comparably high potency, daily dosages between 500 mg and 25 mg, or less, are appropriate. Therefore, it is generally contemplated that among other parameters the patient's particular condition and the potency of the preparation will at least partially determine the frequency of application. For example, where high dosages are to be administered to the patient, more than 3 daily dosages are contemplated, including 4-6 and more. Where low dosages, especially dosages lower than 500 mg/day are contemplated, single, bidaily, or less frequent administrations are appropriate.

Of course it should also be recognized that the form of administration may vary considerably. For example, oral administration need not be limited to a tablet, and alternative oral administrations may include powders, gel-caps, syrups, gels, etc. Where oral administration is not desirable, it is further contemplated that alternative routes are also appropriate, including injections, transdermal, pulmonary or intranasal delivery.

### EXAMPLES

The following examples provide various experimental procedures to make and use contemplated compounds according to the inventive subject matter. Examples 1 and 2 describe basic and improved procedures of producing compositions according to the inventive subject matter, respectively. The biological activity of the compounds isolated according to procedures in Examples 1 and 2 is described in Example 3 and 4, and Example 5 provides experimental support for specific binding of contemplated compounds to thaumatin-like proteins.

### Example 1

Barley grains were malted according to procedures well known in the art of beer brewing (see e.g., Principles of Brewing Science, Second Edition, by George J. Fix; Brewers Publications; ISBN: 0937381748, or The Brewers' Handbook by Ted Goldhammer; KVP Publishers; ISBN: 0967521203). In order to extract soluble substances from the malt and to convert additional insoluble solids into soluble material through controlled enzymatic conversion, a step of mashing was subsequently applied to the ground malt (suspended in water) according to a typical brewer's schedule. The temperature cycles were as follows: Incubation at 40° C. for 60 min, incubation at 50° C. for 60 min, incubation at 60° C. for 60 min, incubation at 72° C. for 60 min, and incubation at 75°-80° C. for 60 min. Soluble portions of samples were separated from husks and other insoluble material and freeze-dried.

The freeze-dried barley extract obtained after mashing at 40° C. served as base for fractionation into its components. A first fractionation was achieved by preparative liquid chromatography using a DEAE-Sephacel column (2.6×20 cm) equilibrated with 50 mM phosphate buffer, pH 7.8. 150 mg of the freeze-dried sample was dissolved in 10 ml of buffer and placed on the column. A linear NaCl-gradient (0-0.5 M) was run at a flow rate of 10 ml/h. Fractions (2 ml each) were collected, and elution was monitored at 280 nm. The DEAE chromatography resulted in four distinct protein peak fractions: I—basic, II—neutral, III- and IV—acidic. Respective peak fractions were collected, desalted and concentrated by membrane ultra-filtration using a membrane cut-off pore size of 1000 Dalton, and concentrated corresponding fractions were checked for their capacity to influence yeast fermentation rate. The basic fraction I produced significant inhibitory effect (i.e., a reduction of the yeast fermentation rate), while the remaining three concentrated fractions were almost inert. As it could later be identified (data not shown), the main proteinaceous component in fraction I represent thaumatin-like proteins. It has been noticed during the membrane ultra-filtration of the pooled protein fractions I-IV (i.e., fractions obtained by ion exchange chromatography), that the filtrate of some fractions contains LMW (low molecular weight) substances with a UV absorbance maximum of approximately 260 nm. These observations prompted us to employ molecular sieving chromatography to separate these LMW substances from proteins in these fractions.

For that purpose, the four separated fractions by DEAE-Sephacel column I-IV were pooled and freeze-dried. Molecular sieving chromatography was performed on Sephadex G-75-50 column (2.8×80 cm) with 50 mM phosphate buffer, pH 7.8, containing 0.5 M NaCl (flow rate—12 ml/h, fractions 2 ml, elution recorded at 260 nm). LMW compounds with an absorbance near 260 eluted at relatively high elution volume. Where the separated fractions were individually subjected to molecular sieving on a Sephadex G-75-50 column, LMW compounds eluted near to the end of the separation, typically between 60th-80th fractions. These fractions were designated GMM-1, GMM-2 and GMM-4, and consist of LMW components.

All of GMM-1, GMM-2 and GMM-4 enhanced yeast fermentation, bound strongly and reversibly to thaumatin-like protein (bind to thaumatin-like proteins at low salt condition and release from thaumatin-like proteins at high salt condition), and reduced elevated blood glucose concentration and elevated blood lipid concentration in human diagnosed with NIDDM.

### Example 2

20 g of malted barley flour was suspended in 80 ml of water and stirred over night at ambient temperature. The suspension was supplemented with 120 ml of 0.8 M NaCl solution and salt extraction was continued for 24 hours with stirring. An aqueous extract was separated from the suspension by vacuum filtration over a cellulose filter pad. Alternatively, citrate or other buffers are also contemplated suitable for preparation of an aqueous extract.

The filtered extract was freeze-dried or vacuum-evaporated. So obtained dry malt extract (yield approx. 12-14 g) contained 5.6 g of NaCl originating from the extracting solvent and a complex mixture of water-soluble barley components. The filtered freeze-dried extract was purified by extraction with two 50 ml portions of warm ethanol under vigorous mixing for two hours. The ethanolic extracts were filtered, combined, and evaporated to an oily residue in vacuum. The oily residue was re-dissolved in 15 ml of water and freeze-dried, resulting in a hard glassy yellowish product in a total amount of approx. 3 g.

The glassy yellowish product enhanced yeast fermentation, bound strongly and reversibly to thaumatin-like protein (bind to thaumatin-like proteins at low salt condition and release from thaumatin-like proteins at high salt condition), and reduced elevated blood glucose concentration and elevated blood lipid concentration in human diagnosed with NIDDM.

Thus, it should be recognized that contemplated compositions comprise a plant seed extract (preferably from Hordeum vulgare), wherein the plant seed is malted (preferably at a temperature between about 30° C. and 65° C.) and the extract is prepared from the malted plant seed using a protocol that includes an aqueous extraction step (e.g., using an aqueous buffer such as a citrate buffer), and that the extract reduces a glucose concentration in an organism when the extract is administered to the organism at a concentration effective to reduce the concentration of glucose.

### Example 3

The biological activity of LMW fractions from Example 1 (GMM-1, GMM-2 and GMM-4) and the glassy yellowish product from Example 2 was monitored by quantification of brewers' yeast fermentation rate under anaerobic conditions using a modified Warburg method (Mirsky, N. et al., J. Inorg. Biochem. 13(1):11-21 (1980), which is incorporated by reference herein.

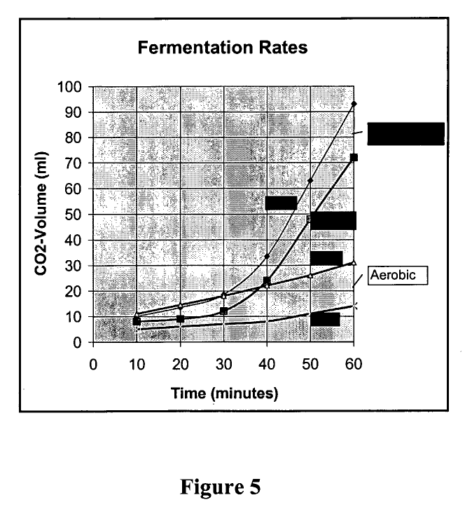

Two grams of wet brewers yeast cells (about 20% dry weight) were suspended in fermentation medium (25 ml of 60 mM phosphate buffer, pH 5.7 and 10 ml of 5% (w/v) glucose solution), and aliquots of the products from example 1 or 2 were added to the fermentation medium for testing. Incubations were carried out in 50 ml fermentation flasks at 25° C. for 60 minutes. The fermentation rates were measured from the volume of generated CO₂. All of the tested LMW fractions or the product from Example 2 showed significant biological activity or bioactivity in that they increased the yeast fermentation rate in the range of about 20-40%. As used herein, a bioactive compound is one that increases or decreases fermentation. In a further experiment, the activity of GMM-2 was checked at aerobic conditions. Despite general restriction of yeast fermentation caused by combined effects of NaCl from buffer and air oxygen (Pasteur effect), the relative amount of generated CO₂ was doubled in comparison to the included control. The comparative results for GMM-2 fraction at anaerobic and aerobic conditions are shown below in FIG. 5. The results conclusively prove modulating activity of the isolated LMW substances on yeast metabolism.

### Example 4

The product obtained in Example 2 was examined for use in humans diagnosed with NIDDM. 25 men were recruited from an outpatient clinic (Endocrinology Department). Mean age within the group was 51 yr, ranging from 36 to 74. Medical records were screened to exclude diabetics taking insulin or oral hypoglycemic agents. All of the subjects agreed to maintain their usual eating habits and health-related behaviors throughout the study. The experimental treatments were run over a period of six month. The participants were instructed to take the preparation in 3 oral daily doses of 1,000 mg each in a tablet form.

All subjects were tested for plasma glucose, glucosylated hemoglobin HbAcl, triglycerides and cholesterol before supplementation and throughout the study at biweekly or monthly intervals depending on type of tests. The subjects were subdivided into groups according to patterns given below:

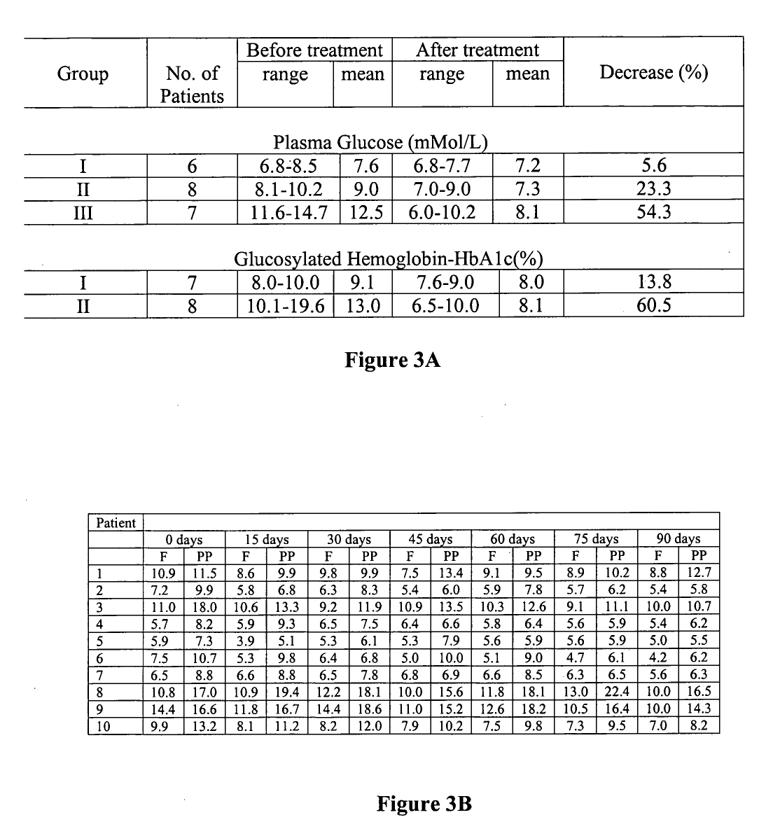

Plasma glucose: According to the plasma glucose levels the subjects were subdivided in three groups for differentiation of the effects: I—up to 8 mMol/L; II—8-10.5 mMol/L and III—above 10.5 mMol/L of plasma glucose concentration. Glycosylated hemoglobin (HbAcl): According to the HbAcl levels the subjects were divided in two groups: I—below 10% and II—above 10% of the modified hemoglobin. The test results related to glycemia, before and after treatment, are shown in FIG. 3A.

A further set of clinical studies was performed with 10 human volunteers following a similar protocol as outlined above. In this second experiment, blood glucose was measured fasting and postprandial over a period of 90 days, and the results are shown in FIG. 3B. As can be clearly seen, administration of contemplated compounds results in a decrease of fasting and/or postprandial blood glucose of at least 5%, more typically of at least 10%, and most typically of at least 20%. Similarly, the levels of glycosylated hemoglobin was reduced after administration of contemplated compounds at least 5%, more typically at least 20%, and most typically at least 50%.

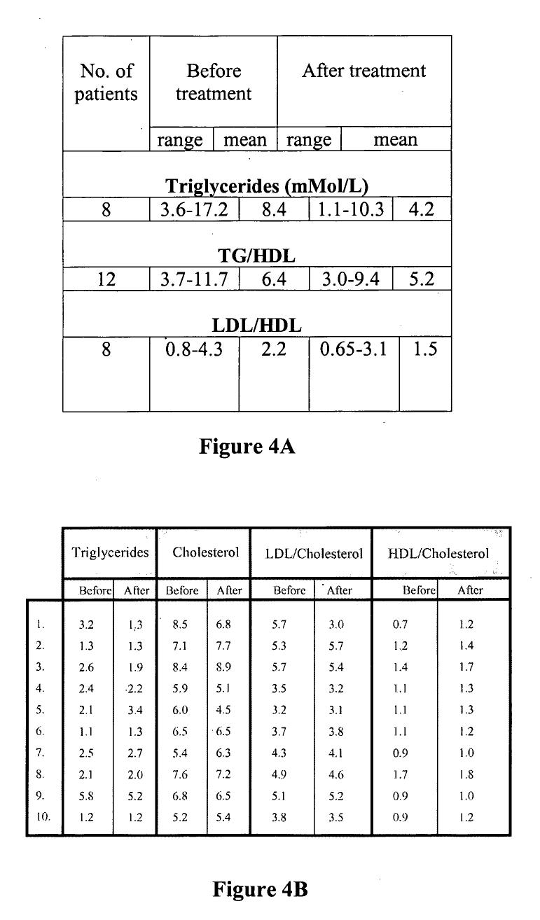

The lipid status of the subjects diagnosed with NIDDM was determined before and after treatment by testing plasma level of triglycerides, and cholesterol (as total, LDL and HDL form). The test results shown in FIGS. 4A and 4B include subjects with disturbed lipid metabolism due to diabetic disease.

The lipid status of the subjects as shown in FIG. 4A includes plasma levels of triglycerides, the ratio of triglycerides over total cholesterol, and the ratio of LDL/HDL. The latter two ratios are known as atherosclerotic risk factors. As can be seen from FIG. 4A, administration of contemplated compounds resulted in a reduction of triglycerides of up to 50%, and a significant reduction of about 1-20% of the ratio of triglycerides to HDL cholesterol, with an even more dramatic reduction of the ratio between LDL to HDL cholesterol (about 40%). The lipid status as shown in FIG. 4B includes further results of ten test patients after administration of contemplated compounds and/or compositions over a period of 90 days.

### Example 5

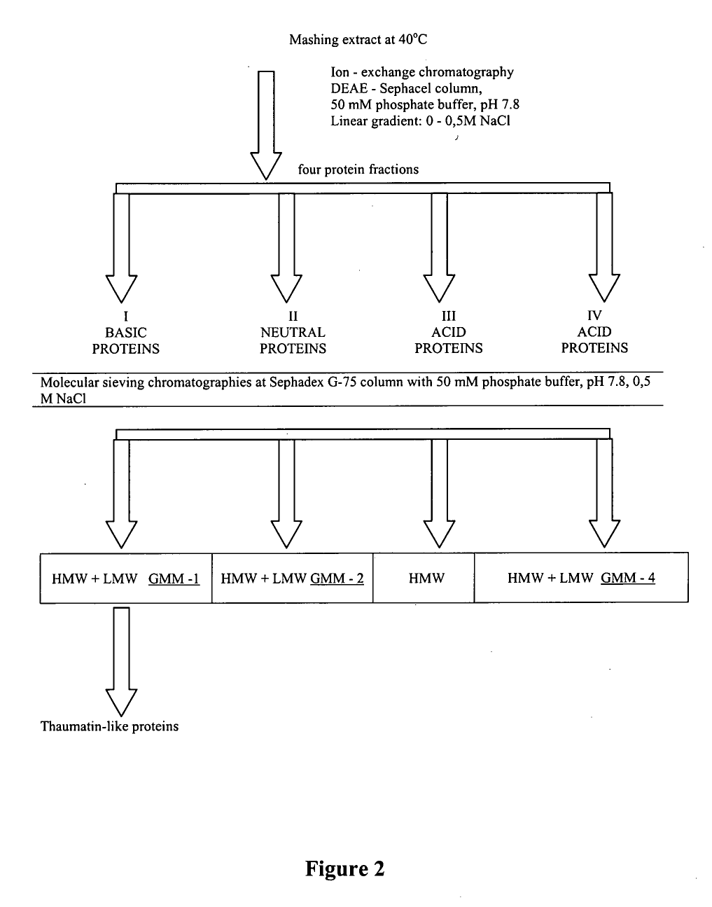

Thaumatin-like proteins were prepared following the procedure as generally outlined in Example 1 and FIG. 2. So isolated thaumatin-like proteins were subjected to repeated molecular sieving in a membrane concentrator using a membrane with a molecular weight cut off of about 1000 Dalton. After a first round of filtration of the protein preparation, 99 ml of buffer (50 mM phosphate-buffer, pH 7.8, 0.5 M NaCl) were added to about 1 ml of retentate (i.e. the thaumatin-like protein fraction), and three subsequent rounds of filtration were performed with the same buffer to remove remaining GMM-compounds (i.e., herein presented compounds that reduce elevated glucose) from the thaumatin-like protein preparation. UV absorbance of the filtrate was monitored at 260 nm and the biological activity of sample volumes from the filtrate was tested according to protocols outlined in Example 3. Such prepared thaumatin-like proteins were desalted by membrane filtration employing NaCl-free buffer (50 mM phosphate buffer, pH 7.8), and further used in the following procedure:

To 1 ml of a desalted thaumatin-like protein solution (10 mg/ml), 1.0 ml of a GMM-1 solution (1mg/ml) was added, and the mixture was incubated at room temperature for 2 hrs. After 2 hrs, 98 ml of 50 mM phosphate buffer, pH 7.8 were added to the mixture and unbound GMM-1 was removed by 3 subsequent rounds of ultrafiltration (each round 1:100 by volume) with buffer.

The thaumatin-like protein with the bound GMM-1 was labeled Sample 1. Sample 1 was then subjected to a molecular sieving chromatography using a Sephadex G-75 column with 50 mM phosphate buffer, pH 7.8, 0.5 M NaCl as solvent, in which a low molecular weight fraction eluted with an absorbance of 260 nm separate from a higher molecular

1-37. (canceled)

38. A method of reducing a concentration of blood glucose in a human, comprising:
preparing a plant extract from hordeum vulgare in a process that includes
(a) malting barley grains, (b) subjecting the malted barley grains to a first salt extraction to obtain a crude extract, (c) refining the crude extract by solvent extraction to form a first refined extract; or
(d) malting barley grains, (e) subjecting a soluble fraction of the malted barley grains to a chromatographic step to obtain a crude extract that includes a thaumatin-like protein, (f) refining the crude extract by size fractionation to form a second refined extract;
wherein the steps (a)-(c) or (d)-(f) are selected such that the first or second refined extract provide a composition that includes a compound that binds to a thaumatin-like protein; and
administering the composition to the organism in a dosage effective to decrease the concentration of glucose.

39. The method of claim 38 further comprising a step of removing the solvent to thereby form a concentrate.

40. The method of claim 38 wherein the composition has a molecular weight of no more than 1000 Da.

41. The method of claim 38 wherein the composition has an UV absorption maximum of about 260 nm

42. The method of claim 38 wherein the composition is orally administered in a dosage that is effective to reduce glycosylated hemoglobin at least 5%, to reduce plasma glucose at least 10%, to reduce plasma triglycerides between 4% to 50%, and to reduce a LDL to HDL cholesterol ratio up to 40% in the human.

43. The method of claim 38 wherein the human is diagnosed with non-insulin dependent diabetes mellitus.

44. The method of claim 38 further comprising a step of adding a tocol to the composition.

45. The method of claim 38 wherein the process includes at least one procedure selected from the group consisting of mashing, a buffer extraction, ethanol extraction, anion exchange chromatography, and molecular sieving.

46. The method of claim 38 wherein the composition further reduces a blood concentration of a lipid at the dosage effective to decrease the concentration of glucose.

47. The method of claim 46 wherein the lipid is selected from the group consisting of a triglyceride, a fatty acid, and a LDL-cholesterol.

48. The method of claim 38 wherein the malting includes a step of malting at a temperature between 30° C. and 65° C.

49. The method of claim 38 wherein the salt extraction includes a step of adding NaCl at a concentration of greater than 0.5M to form a salt extract.

50. The method of claim 38 wherein the solvent extraction comprises ethanol extraction.

51. The method of claim 38 wherein the chromatographic step comprises anion exchange chromatography.

52. The method of claim 38 wherein the size fractionation comprises molecular sieving.
